(19) United States
(12) Patent Application Publication
(10) Pub. No.: US 2006/0079447 A1
(43) Pub. Date: Apr. 13, 2006

(54) Stabilized A-beta protofibrillar aggregates

(76) Inventor: Ronald B. Wetzel, Knoxville, TN (US)

Correspondence Address: FENWICK & WEST LLP, SILICON VALLEY CENTER, 801 CALIFORNIA STREET, MOUNTAIN VIEW, CA, 94041, US

(21) Appl. No.: 11/245,501

(22) Filed: Oct. 7, 2005

Related U.S. Application Data
(60) Provisional application No. 60/617,114, filed on Oct. 8, 2004.
(60) Provisional application No. 60/674,101, filed on Apr. 22, 2005.

(51) Int. Cl. A61K 38/17; A61K 31/4164

(52) U.S. Cl. 514/2; 514/396

(57) ABSTRACT

Described and claimed herein are methods for producing chemically stabilized Aβ protofibrillar aggregates, and compositions made therefrom. Compositions produced are useful in facilitating detailed studies of Aβ protofibril structure, fibril formation, and progression of Aβ related diseases, e.g., Alzheimers disease. In addition, chemically stabilized protofibrillar structures can be used as tools to generate and/or screen for antibodies specific for protofibrils. Antibodies specific for protofibrils can be used as diagnostic tools or as therapeutics in the diagnosis or treatment of, e.g., Alzheimer's disease. Finally, chemically stabilized protofibrillar structures can be used in the preparation of therapeutic or prophylactic vaccines.

### CROSS REFERENCE TO RELATED APPLICATIONS

This application claims the benefit of U.S. Provisional Application No. 60/617,114, filed Oct. 7, 2004 and U.S. Provisional Application No. 60/674,101, filed Apr. 22, 2005, the entire disclosures of which are hereby incorporated by reference in their entirety for all purposes.

### STATEMENT REGARDING FEDERALLY SPONSORED RESEARCH OR DEVELOPMENT

The U.S. Government has certain rights in this invention pursuant to Grant Nos. R01AG18416 and R01 AG18927 awarded by the National Institutes of Health.

### BACKGROUND OF THE INVENTION

1. Field of the Invention

The invention relates to methods for producing stabilized Aβ protofibrillar aggregates using stabilizing compounds, e.g., small molecules such as CLC; the stabilized Aβ protofibrillar aggregates produced by the methods; and use of the methods and aggregates for screening for stabilizing compound and specific binding reagents, e.g., antibodies. The invention is useful for the study of Aβ structure and function for research and development of vaccines and therapeutics for treatment of Alzheimer's disease.

2. Description of the Related Art

Protofibrils and oligomers are metastable peptide assemblies observed during the growth of amyloid fibrils by a number of peptides, including the Alzheimer's amyloid plaque peptide Aβ (1-3). These oligomeric assemblies are important for at least two reasons. First, it is now believed that such forms, rather than mature fibrils, may be the cytotoxic agents responsible for some amyloid-associated disorders like Alzheimer's and Parkinson's diseases (4, 5). Second, it has been postulated that these structures may be intimately involved in the amyloid fibril assembly mechanism, both in amyloid nucleation and fibril elongation (2, 3, 6).

In Aβ assembly, protofibrils are generally observed during the lag phase of spontaneous amyloid growth at relatively high Aβ concentrations. Isolated Aβ protofibrils incubated in buffer tend to dissociate (7), suggesting a fundamental instability incompatible with many biophysical techniques. Working against this instability, Kheterpal et al. used hydrogen-deuterium exchange (HX) to show that Aβ protofibrils contain a subset of highly protected, presumably H-bonded, backbone amide protons (8). Similarly, photo-crosslinking has been used to characterize small, metastable oligomeric forms of AP that may represent protofibril assembly intermediates and/or substructures (9). More detailed characterization of such structures, however, is made difficult by their metastable, transient nature.

Klein (US 2003/0068316) describes soluble, globular oligomeric Aβ structures called amyloid beta-derived diffusible ligands (ADDLs) and antibodies specific for ADDLs. The ADDLs are formed by diluting a solution of Aβ (1-42) in cold tissue culture solution. Under highly defined conditions, conversion of ADDLs to protofibrils or fibrils is suppressed. The size range of ADDLs tends to be small as measured by AFM, from about 4.7 to 11.0 nm, and from 3 to 24 monomers. Aβ (1-40) cannot form stable ADDLs. ADDLs have not been complexed with a small molecule, and their long range stability is unknown.

Glabe (Kayed et al (2003) Science 300:486) describes molecular mimics of soluble Aβ oligomers and antibodies specific for these molecular mimics. The molecular mimics are formed by linking the C-termini of multiple Aβ peptides to gold beads. These covalent oligomers were used in rabbits to generate anti-sera specific for non-fibrillar Aβ aggregate intermediates.

Bohrman (Bohrmann, B., et al (2000) J Struct Biol 130, 232-46) describes formation of nonfibrillar, polymeric sheet intermediates using Aβ (1-42) and a compound Ro 90-7501. The compound retarded but did not block formation of mature fibrils.

Many attempts have been made to identify compounds that alter the course of the amyloid formation reaction by Aβ and other proteins (10). Although there are many reports on the identification of such compounds, little is known about the mechanism by which they affect fibrillogenesis. Such a compound, and Aβ altered by such a compound, would be a useful tools in addressing aspects of the fibril assembly mechanism. In addition, compounds that influence aggregate assembly pathways could be useful in design or screening for therapeutic agents for treatment of Aβ related disease, e.g., Alzheimer's disease.

### SUMMARY OF THE INVENTION

Described herein is chemical biology approach to structural analysis of Aβ protofibrils. Library screening yielded several molecules that stimulate Aβ aggregation. One of these compounds, calmidazolium chloride (CLC), rapidly and efficiently converts Aβ(1-40) monomers into clusters of protofibrils, e.g., stabilized Aβ protofibrillar aggregates. As monitored by electron microscopy, these stabilized Aβ protofibrillar aggregates persist for days when incubated in phosphate buffered saline (PBS) at 37° C., with a slow transition to fibrillar structures apparent only after several weeks. Like normal protofibrils, the stabilized Aβ protofibrillar aggregates exhibit a low thioflavin T response. Like Aβ fibrils, the stabilized Aβ protofibrillar aggregates bind the anti-amyloid antibody WO1. The stabilized Aβ protofibrillar aggregates exhibit the same protection from hydrogen-deuterium exchange as do protofibrils isolated from a spontaneous Aβ fibril formation reaction: about 12 of the 39 Aβ(1-40) backbone amide protons are protected from exchange in the protofibril, compared to about twice that number in amyloid fibrils. Scanning proline mutagenesis analysis shows that the Aβ molecule in these stabilized Aβ protofibrillar aggregates exhibits the same flexible N- and C-termini as do mature amyloid fibrils. The major difference in Aβ conformation between fibrils and stabilized Aβ protofibrillar aggregates is added structural definition in the 22-29 segment in the fibril. Besides aiding structural analysis, compounds capable of facilitating oligomer and protofibril formation have therapeutic potential, and/or can be used to screen for therapeutics. In addition, stabilized Aβ protofibrillar aggregates can be used to screen for antibodies useful for both research and therapeutics.

Accordingly, one aspect of the invention are methods for producing a stabilized Aβ protofibrillar aggregate comprising contacting a plurality of Aβ peptide molecules with a stabilizing compound, wherein said stabilizing compound stabilizes a protofibrillar aggregate form of the peptide as compared to the peptide not in contact with the stabilizing compound. In some embodiments, stabilization is measured, e.g. using a sedimentation assay comprising centrifugation and analytical HPLC. The structure, e.g., physical characteristics, of the stabilized Aβ protofibrillar aggregates is different from that of amyloid fibrils as assayed by, e.g., electron microscopy, thioflavin T response, ability to seed aggregation in the presence of Aβ peptide, and protection of backbone amide hydrogens from hydrogen-deuterium exchange. The Aβ peptide can be any version of Aβ peptide capable of forming a stabilized Aβ protofibrillar aggregate, including but not limited to Aβ (1-40) or Aβ (1-42). In some embodiments, prior to contact with the stabilizing compound, no seeding amyloid fibril is present.

Numerous stabilizing compounds are contemplated, e.g., small molecules and the like. In some embodiments, the stabilizing compound is calmidazolium chloride (CLC). Accordingly, in one aspect of the invention, the method for producing a stabilized Aβ protofibrillar aggregate includes contacting Aβ (1-40) peptide with calmidazolium chloride (CLC).

Compositions, e.g., stabilized Aβ protofibrillar aggregates, produced by the methods of the invention are also disclosed and claimed. In one embodiment, the composition includes Aβ(1-40) protofibrillar aggregates stabilized by CLC. Pharmaceutical compositions including stabilized Aβ protofibrillar aggregates of the invention are one embodiment.

Also included in the invention are methods of screening for stabilizing compounds. The method has the steps of contacting an Aβ peptide with a test compound and detecting the presence or absence of a stabilized Aβ protofibrillar aggregate, wherein the presence of the stabilized Aβ protofibrillar aggregate indicates that the test compound is a stabilizing compound. The test compound can be any small molecule. The screening method can be performed in the presence or absence of seeding amyloid fibril.

The invention also provides methods for using the compositions of the invention to screen for binding reagents specific for a stabilized protofibrillar aggregate form of an Aβ peptide, e.g., contacting a test binding reagent with the stabilized Aβ protofibrillar aggregate and detecting the presence or absence of specific binding to the stabilized Aβ protofibrillar aggregate, wherein specific binding indicates the test binding reagent is a binding reagent specific for a stabilized Aβ protofibrillar aggregate. The test binding reagent can be an antibody, any other polypeptide or a nucleic acid. In one aspect of the screening method, the Aβ protofibrillar aggregate is immobilized to, e.g., a microtiter plate or a solid surface or a bead.

The invention also provides a method of generating an immune response in an animal comprising administering an effective amount of a stabilized Aβ protofibrillar aggregate. In one embodiment, the stabilized Aβ protofibrillar aggregate is Aβ (1-40) stabilized with CLC. The animal can be, e.g., a rabbit or a human. A further aspect of the invention is isolating antibodies specific for stabilized Aβ protofibrillar aggregates using the method of generating an immune response.

### BRIEF DESCRIPTION OF THE SEVERAL VIEWS OF THE DRAWINGS

FIG. 1 is a graph illustrating screening of the LOPAC library of 640 compounds for inhibition of Aβ elongation. Inset: structure of CLC.

FIG. 2a illustrates the CLC effect on Aβ deposition in microplate assay: CLC present in wells with immobilized fibrils (♦), CLC present in wells with no fibrils (▪), and fibril in wells but no CLC (▴). FIG. 2b is a dose-response curve for CLC effect on Aβ deposition FIG. 2c illustrates the seeding abilities of APβ aggregates grown in the presence of 100 μM CLC for 2 days (□) and 14 days (▴), or aggregates grown in the absence of CLC for 2 days (●) and 14 days (♦)

FIG. 3a illustrates the results of a solution phase aggregation reaction of Aβ(1-40) monitored by ThT (▴,♦) and HPLC of centrifugation supernatants (●,▪). Disaggregated Aβ(1-40) was incubated alone (▪,♦) or with 100 μM CLC (●,570 ). FIG. 3b illustrates the a solution phase time course of reaction of 100 μM CLC with Aβ(1-40) variants. ⋄, wt; ●, F4P; Δ, V18P; ◯, I31P; □, F19P/I32P. FIG. 3c illustrates antibody binding to Aβ(1-40) aggregates. WO1 binding to Aβ amyloid fibrils (▪), CLC-Aβ aggregates (●), and a no aggregate control (♦); control IgM antibody binding to Aβ amyloid fibrils (□),CLC-Aβ aggregates (◯), and a no aggregate control (⋄).

FIG. 4 is an electron micrographs of aliquots of Aβ(1-40) aggregation reactions in the absence (a, 2 days; b, 14 days) or presence (c, 2 days; d, 14 days) of 100 μM CLC. Aggregates were adsorbed onto carbon coated copper grids and negatively stained with either 0.5% uranyl acetate (a inset and b) or 1% potassium phosphotungstate, and photographed on a Hitachi H-800 EM. The inset in panel a is adapted from FIG. 1 of reference (8).

FIG. 5 illustrates hydrogen-deuterium exchange of various Aβ(1-40) aggregates, corrected for back and forward exchange (17) into side chains only (a) or into side chains plus main chain (b). Fitted curves for repeated runs on monomers (— -- — --) and fibrils (______). ▪, averaged data for exchange into protofibrils taken from reference8; ▴, exchange of Aβ(1-40) fibrils produced in experiment described here (FIG. 4b); ♦, 2 day product of incubation of Aβ(1-40) with 100 μM CLC.

FIG. 6 illustrates Aβ(1-40) concentration in centrifugation supernatants after incubating various mutant peptides (a) with 100 μM CLC for two days or (b) with Aβ(1-40) seed fibrils until equilibrium is reached (data from reference 15). More positive values signify lower aggregate stability. Codes for double proline mutants: P2=23, 30; P4=9, 23, 30, 37; β2=19, 32.

FIG. 7 illustrates preparation and characterization of Aβ(1-42) CLC aggregates. (a) About 7 μM disaggregated Aβ(1-42) was incubated in PBS at 37° C. with 100 μM CLC. Aliquots were monitored for the amount of monomeric peptide left in solution after centrifugation (▪) and the amount of thioflavin T signal generated (□). As a control, the same amount of peptide was allowed to make amyloid fibrils and the ThT signal monitored over time (Δ). (b) Microtiter plate wells were coated with Aβ(1-42)-aggregates and then incubated with biotinyl-Aβ(1-40) to determine the ability of the aggregate to stimulate elongation (amyloid fibrils, ●; CLC aggregates, ▪).

FIG. 8 illustrates binding of antibodies to immobilized Aβ(1-42) amyloid fibrils (●), Aβ(1-42)-CLC aggregates (▪), and bovine serum albumin blocking agent control (⋄). (a) WO1; (b) Chemicon MAb1560, the 6E10 IgG that binds to the Aβ N-terminus; (c) Calbiochem mouse myeloma IgM (catalog 401925). Note y-scale difference in part c. All data except for the BSA control curve data represents crude data from which BSA binding data was subtracted (BSA is the blocking agent present in all experiments).

FIG. 9 illustrates the structures of the seven compounds identified in the original LOPAC screen. A—Amiloride HCl. Initial stimulation observed=96%; EC₅₀≧38.5 μM. B—Phenamil methane sulfonate. Initial stimulation observed=242%; EC₅₀≧35.5 μM. C—Calmidazolium chloride. Initial stimulation observed=598%; EC₅₀≧21.4 μM. D—Naftopidil dihydrochloride. Initial stimulation observed=150%; EC₅₀≧36.2 μM. E—L-α-methyl DOPA. Initial stimulation observed=127%; EC₅₀≧34.2 μM. F—5-(nonyloxy)-tryptamine hydrogen oxalate. Initial stimulation observed=136%; EC₅₀≧30.3 μM. G—NPC15437. Initial Stimulation observed=149%; EC₅₀>27.2 μM.

### DETAILED DESCRIPTION OF THE INVENTION

Described is a screening assay for Aβ elongation to identify modifiers of Aβ aggregation. Stabilizing compounds were identified that function by stabilizing Aβ protofibrillar aggregates. One compound, calmidazolium chloride (CLC), was particularly effective. The stabilized Aβ protofibrillar aggregates can used for studying Aβ structure and properties; they can also be used for development and screening of therapeutics; e.g., vaccines and therapeutics for amyloidal diseases, e.g., Alzheimer's.

Definitions

Terms used in the claims and specification are defined as set forth below unless otherwise specified.

Stabilized Aβ protofibrillar aggregate refers to an oligomeric, aggregated, protofibrillar Aβ structure that is produced by contacting Aβ peptide with a stabilizing compound. Stabilized Aβ protofibrillar aggregates are significantly retarded from progressing to a mature amyloid fibril form. Stabilized Aβ protofibrillar aggregates exhibit at least one of physical properties described in detail herein.

Stabilizing compound refers to a compound, e.g., a small molecule, that stabilizes an protofibrillar aggregated form of Aβ peptide, e.g., retard progression to mature amyloid.

Binding reagent refers to a reagent that specifically binds to a target, e.g., a stabilized Aβ protofibrillar aggregate. Binding reagents are generally macromolecules and include but are not limited to antibodies, other polypeptides (e.g., ankyrins), and nucleic acids (e.g., RNA aptimers). Antibody as used herein includes any protein polypeptide having at least a portion of an immunoglobulin molecule, such as, but not limited to, at least one complementarity determining region (CDR) of a heavy or light chain or a ligand binding portion thereof, a heavy chain or light chain variable region, a heavy chain or light chain constant region, a framework region, or any portion thereof.

An animal includes but not limited to, a human, a mouse, a rabbit, a rat, a rodent, a goat, a primate, or any combination thereof, and the like.

Abbreviations used in this application include the following: calmidazolium chloride, CLC; thioflavin T, ThT; PBSA, phosphate-buffered saline containing 0.05% sodium azide; HX, hydrogen-deuterium exchange; EM, electron microscopy.

Methods for Producing Stabilized Protofibrillar Aggregates

Disclosed herein are methods for producing a stabilized Aβ protofibrillar aggregate. The method includes contacting a plurality of Aβ peptide with a stabilizing compound. The Aβ peptide is in an environment that promotes formation of protofibrillar aggregates, e.g., the APβ(1-40) microplate elongation assay as described in O'Nuallain (O'Nuallain et al (2004) J Biol Chem 279, 17490-17499). In other embodiments, the Aβ peptides are aggregated in solution phase, e.g., incubated in 50 μM PBSA at 37° C. and described in more detail herein. Other reaction conditions for aggregation can be readily determined by one of skill in the art and depend on, e.g., the specific Aβ peptide and stabilizing compound used; parameters that can be optimized include, e.g., temperature, salt, and concentration of Aβ peptide and stabilizing compound.

The method for producing a stabilized Aβ protofibrillar aggregate can be performed either in the presence or absence of seeding Aβ amyloid fibril. In a preferred embodiment, the method is performed in the absence of Aβ amyloid fibril.

Stabilization and physical properties of protofibrillar aggregates

Stabilization and fibrillar properties of stabilized Aβ protofibrillar aggregates are measured by any number of techniques known to one of skill in the art and include, e.g., electron microscopy, thioflavin T response, protection of backbone amides from hydrogen-deuterium exchange, ability to seed aggregate growth by monomer addition, and proline scanning mutagenesis. All of these techniques are described in detail in the Examples section. Other methods include, e.g., turbidity and light scattering assays that detect particle formation, sedimentation assays combining centrifugation with measurements of the amount of material pelleted, and/or the amount of material remaining unpelleted. Some of these methods are best used to demonstrate the formation of aggregates from momomeric, soluble protein. Others of these methods are best used to conduct detailed structural investigations of the aggregates in order to distinguish one aggregate type from another. One of skill will appreciate that the appropriate assay is chosen depending on the property to be measured.

In summary, the methods measure the stabilization and fibrillar properties of stabilized Aβ protofibrillar aggregates as compared to Aβ peptide not in contact with a stabilizing compound and/or compared to protofibrils or fibrils. In some embodiments, the stabilized Aβ protofibrillar aggregates of the invention include one of the properties described herein. In other embodiments, stabilized Aβ protofibrillar aggregates include more than one of the properties described herein.

In one embodiment, when observed using electron microscopy (EM), stabilized Aβ protofibrillar aggregates produced by the methods of the invention resemble protofibrils. For example, stabilized Aβ protofibrillar aggregates of the invention can exhibit conglomerates that can be seen to be composed of smaller sub-structures that in the EM resemble protofibrils, and that, when rigorously vortexed, yield EM images showing a dense lawn of structures resembling protofibrils.

In another embodiment, stabilized Aβ protofibrillar aggregates produced by the methods of the invention closely resemble protofibrils as determined by biochemical analysis, e.g., the stabilized Aβ protofibrillar aggregates are not effective seeds for elongation by monomeric Aβ(1-40) in a plate aggregation assay in contrast to mature Aβ amyloid fibrils.

In further embodiments, stabilized Aβ protofibrillar aggregates are similar to Aβ protofibrils in their HX behavior, e.g., fewer backbone amide hydrogens are protected compared to mature amyloid fibrils. In some embodiments, the number of backbone amide hydrogens protected is identical to that seen previously for authentic Aβ protofibrils isolated from normal fibril formation reactions.

Still another characteristic of stabilized Aβ protofibrillar aggregates of the invention is binding the antibody W01 (O'Nuallain et al. (2002) Proc Natl Acad Sci U S A 99, 1485-1490). The stabilized Aβ protofibrillar aggregates bind W01 with reduced affinity compared to amyloid fibrils. In some embodiments, stabilized Aβ protofibrillar aggregates bind W01 with about 10-fold reduced affinity compared to binding by amyloid fibrils.

A further characteristic of stabilized Aβ protofibrillar aggregates is measured by the effect of scanning proline mutagenesis on aggregation reactions compared to the effect of the same proline substitutions on aggregation reactions of amyloid fibrils. In one embodiment, for both stabilized Aβ protofibrillar aggregates and amyloid fibrils, proline replacement of residues in the N-terminal region is negligibly destabilizing, while proline substitution at many other positions in the 15-40 segment, especially the hydrophobic clusters at positions 16-21 and 31-36, are destabilizing. Proline replacements at positions 9, 23, 30 and 37 destabilizes stabilized Aβ protofibrillar aggregates, in contrast to amyloid fibrils. Prolines at residues 24-28 destabilize amyloid fibril formation, in contrast to stabilized Aβ protofibrillar aggregates.

Aβ Peptides

As is well known to one of skill in the art, Aβ peptide is derived from proteolytic cleavage of a membrane protein, amyloid precursor protein (APP). The nucleic acid and protein sequences are known, and the peptide can be produced using standard recombinant and chemical methods well known in the art. As used herein, the term “Aβ peptide” refers to any Aβ peptide that can be used for the methods of the invention, e.g., that can form a stabilized Aβ protofibrillar aggregates. For example, in addition to Aβ (1-40), Aβ is also known in other lengths, e.g., Aβ (1-42) and Aβ (1-43). Aβ peptide can be truncated at the amino terminus, e.g., Aβ (3-42) and the like. Likewise, truncations or extensions at the C-terminus of Aβ are also encompassed by the term Aβ. Likewise, covalently crosslinked versions of Aβ, for example via to Cys residues introduced into the peptide, are also encompassed by the term Aβ. Chemically modified versions are also contemplated. Mutant version of the Aβ peptide, e.g., replacements of residues 24, 25, or 26 with proline, are also encompassed by the term Aβ peptide. In addition, version of Aβ peptide containing one or more of the mutations linked to familial AD (e.g., the Swedish mutation, etc.) are also contemplated.

Unless otherwise specifically noted, the term is meant to include any of the variants described herein. A specific notation is used when referring to a specific variant, e.g., the term Aβ (1-40 refers to an Aβ peptide with a specific length of 40 amino acids; SEQ ID NO:1 refers to Aβ peptide with the amino acid sequence disclosed in SEQ ID NO: 1.

Stabilizing Compounds

As described herein, the methods of the invention use a stabilizing compound for producing stabilized Aβ protofibrillar aggregates. In one embodiment, the stabilizing compound is a small molecule. A small molecule is a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In further embodiments, stabilizing compounds of the invention are those identified from the LOPAC (Library of Pharmacologically Active Compounds) chemical library from Sigma. In one embodiment, the stabilizing compound includes at least one of naftopidil dihydrochloride, dihydrochloride, calmidazolium chloride (CLC), 5-(nonoxyl)-tryptamine hydrogen oxalate, and L-α-methyl DOPA. In a preferred embodiment, the stabilizing compound is calmidazolium chloride (CLC).

It is to be understood that molecular embodiments of stabilizing compounds of the present invention, e.g., CLC, can possess one or more chiral centers and each center may exist in the R or S configuration. The present invention includes all diastereomeric, enantiomeric, and epimeric forms as well as the mixtures thereof. Stereoisomers can be obtained, if desired, by methods known in the art as, for example, the separation of stereoisomers by chiral chromatographic columns. Additionally, the compounds of the present invention can exist as geometric isomers. The present invention includes all cis, trans, syn, anti, entgegen (E), and zusammen (Z) isomers as well as the mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem, Rev, 1996, 96, 3147-3176 and references cited therein.

In addition, the stabilizing compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The stabilizing compounds of the invention, especially when used in the invention methods and compositions, may be “conjugated”—that is they may be coupled to additional moieties that do not destroy their ability to stabilize Aβ protofibrillar aggregates. For example, the compounds might be coupled to a label such as a radioactive label, a fluorescent label and the like, or may be coupled to targeting agents such as antibodies or fragments, or to fragments to aid purification such as FLAG or a histidine tag. The compounds may also be coupled to specific binding partners such as biotin for use in assay procedures or to moieties that alter their biological half-lives such as polyethylene glycol. Thus, the methods of the invention employ the invention compounds per se as well as conjugates thereof.

The concentration of stabilizing compound used in the methods of the invention depend on the individual stabilizing compound and reaction conditions used, and can readily be determined by one of skill in the art. Preferably, the concentrations used are in the micromolar range. In one embodiment, the concentration of stabilizing compound used is from 1-200 μM; in further embodiments, the stabilizing compound concentration is, e.g., 1-100 μM or 10-100 μM or 50-100 μM. In one embodiment, the stabilizing compound concentration is about 10 μM.

Alternatively, screening can be used to screen a test compound or, e.g., a library of test compounds, for a stabilizing compound. The term test compound refers to a compound that is to be screened in one or more of the assays described herein. The compound can be virtually any chemical compound, e.g., any small molecule. It can exist as a single isolated compound or can be a member of a chemical (e.g., combinatorial) library. In a particularly preferred embodiment, the test compound will be a small organic molecule.

Pharmaceutical Compositions of the Invention

The stabilized protofibrillar aggregates of the invention can be formulated in pharmaceutical compositions. These compositions can comprise, in addition to one or more of the stabilized protofibrillar aggregates, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

A pharmaceutical composition compound according to the present invention is administered in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A pharmaceutical composition can be administered alone or in combination with other treatments, either simultaneously or sequentially dependent upon the condition to be treated.

Methods of Screening for Binding Reagents to Stabilized Protofibrillar Aggregates

Also contemplated are use of the stabilized Aβ protofibrillar aggregates produced by the methods of the invention to screen for binding reagents, e.g., antibodies and the like, that specifically bind to stabilized Aβ protofibrillar aggregates. Binding reagents identified by a screen are useful for studying Aβ fibril formation and Alzheimer's disease. In addition, the stabilized Aβ protofibrillar aggregate specific binding reagents can be used in, e.g., developing therapeutics and prophylactic vaccines.

Screening of any number of macromolecules that are capable of binding to a stabilized Aβ protofibrillar aggregates are contemplated. Examples include but are not limited to antibodies, other polypeptides, e.g., ankyrins, and nucleic acids, e.g., RNA aptimers.

The term “specifically binds,” as used herein, when referring to a binding reagent (e.g., protein, nucleic acid, antibody, etc.), refers to a binding reaction that is determinative of the presence binding reagent in a heterogeneous population of molecules (e.g., proteins and other biologics). Thus, under designated conditions (e.g., immunoassay conditions in the case of an antibody), the binding reagent binds to its target molecule, e.g., stabilized Aβ protofibrillar aggregate, and does not bind in a significant amount to other molecules present in the sample.

In one embodiment, the invention provides a method to screens for antibodies specific for stabilized Aβ protofibrillar aggregates. An antibody refers to a protein consisting of one or more polypeptides substantially encoded by immunoglobulin genes or fragments of immunoglobulin genes. Antibodies exist as intact immunoglobulins or as a number of well characterized fragments produced by digestion with various peptidases, e.g., Fabs. While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies.

Antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. In one embodiment, the antibodies include those identified using phage display techniques (e.g., scFv, Fv, Fab and disulfide linked Fv (Reiter et al. (1995) Protein Eng. 8: 1323-1331)).

General methods of screening antibodies for specific binding to a target are well known to one of skill in the art and are meant to be included in the methods of screening for binding reagents specific for stabilized Aβ protofibrillar aggregates. Methods include the antibody binding assay described herein (in Examples) and, e.g., ELISAs, Western blots, or immunohistochemistry that are routinely used to select antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

In one embodiment, test antibodies are screened for binding to immobilized stabilized Aβ protofibrillar aggregates on 96-well microtiter plate.

Methods of Generating an Immune Response

The invention also provides methods of generating an immune response in an animal by administering an effective amount of a stabilized Aβ protofibrillar aggregate. The methods for generating an immune response are useful for, e.g., generating antibodies (e.g., polyclonal, monoclonal, recombinant, and humanized antibodies) that specifically bind to stabilized Aβ protofibrillar aggregates. The immune response can be generated in any animal, human or non-human; non-human animals include those useful for research and development, e.g., mouse, rabbit, sheep, rat, guinea pig, goat, and the like.

An effective amount of stabilized Aβ protofibrillar aggregate immunogen is the amount that will generate an immune response, e.g., generate anti-stabilized Aβ protofibrillar aggregate antibodies. Reactivity of anti-stabilized Aβ protofibrillar aggregate antibodies against the target stabilized Aβ protofibrillar aggregate can be established by a number of well known means, including Western blot, immunoprecipitation, ELISA, and FACS analyses using stabilized Aβ protofibrillar aggregate.

The most preferred antibodies generated using the method will selectively bind to stabilized Aβ protofibrillar aggregates and will not bind (or will bind weakly) to other proteins, e.g., Aβ peptide (monomer) and/or Aβ amyloid fibrils. Anti-stabilized Aβ protofibrillar aggregate antibodies that are particularly contemplated include monoclonal and polyclonal antibodies as well as fragments thereof (including, e.g., recombinant proteins) containing the antigen binding domain and/or one or more complement determining regions of these antibodies. These antibodies can be from any source, e.g., mouse, rabbit, sheep, goat, rat, dog, cat, pig, horse, or human.

Stabilized Aβ protofibrillar aggregate antibodies generated using the methods of the invention are useful in, e.g., various immunological assays for the study of Aβ structure and function and studying Alzheimers disease. Such immunological assays generally comprise one or more stabilized Aβ protofibrillar aggregate antibody capable of recognizing and binding a stabilized Aβ protofibrillar aggregate, and include various immunological assay formats well known in the art, including but not limited to various types of precipitation, agglutination, complement fixation, radioimmunoassays (RIA), enzyme-linked immunosorbent assays (ELISA), enzyme-linked immunofluorescent assays (ELIFA) (H. Liu et al. Cancer Research 58: 4055-4060 (1998), immunohistochemical analysis and the like. In addition, immunological imaging methods using stabilized Aβ protofibrillar aggregate antibodies generated using the methods of the invention are can be clinically useful in the detection, monitoring, and prognosis of Alzheimers disease. Stabilized Aβ protofibrillar aggregate antibodies generated using the methods of the invention can also be used in methods for purifying stabilized Aβ protofibrillar aggregates, and to identify and localize Aβ protofibrillar structures in cells and tissue.

Various methods for the preparation of antibodies are well known in the art and are included in the methods of generating an immune response as claimed herein. For example, antibodies can be prepared by immunizing a suitable mammalian host using a stabilized Aβ protofibrillar aggregate in isolated or immunoconjugated form (Harlow, Antibodies, Cold Spring Harbor Press, NY (1989)). In addition, fusion proteins of Aβ, such as a GST-fusion proteins, can be used to produce stabilized Aβ protofibrillar aggregates, and used for immunization.

Methods for preparing a protein for use as an immunogen and for preparing immunogenic conjugates of a protein with a carrier such as BSA, KLH, or other carrier proteins are well known in the art. In some circumstances, direct conjugation using, for example, carbodiimide reagents may be used; in other instances linking reagents such as those supplied by Pierce Chemical Co., Rockford, Ill., may be effective. Administration of a stabilized Aβ protofibrillar aggregate immunogen is conducted generally by injection over a suitable time period and with use of a suitable adjuvant, as is generally understood in the art. During the immunization schedule, titers of antibodies can be taken to determine adequacy of antibody formation.

The method of generating an immune response can be used to generate polyclonal antibodies using, e.g., rabbit, goats, and/or sheep immunized with stabilized Aβ protofibrillar aggregate. Generally, sera is isolated form immunized animals and used directly or, alternatively, anti-stabilized Aβ protofibrillar aggregate polyclonal antibody is isolated using affinity purification techniques.

The method for generating an immune response can also be used to generate a monoclonal antibody, using, e.g., spleen of a sacrificed animal immunized with stabilized Aβ protofibrillar aggregate. Immortalized cell lines which secrete a desired monoclonal antibody can be prepared using the standard method of Kohler and Milstein or modifications which effect immortalization of lymphocytes or spleen cells, as is generally known. The immortalized cell lines secreting the desired antibodies are screened by immunoassay in which the antigen is the stabilized Aβ protofibrillar aggregate. When the appropriate immortalized cell culture secreting the desired antibody is identified, the cells can be cultured either in vitro or by production in ascites fluid.

In alternative embodiment, the method of generating an immune response is used to produce antibodies or fragments by recombinant means, e.g., isolating the spleen of an immunized animal and creating a Vh-Vl library. For example, fully human anti-stabilized Aβ protofibrillar aggregate monoclonal antibodies may be generated using cloning technologies employing large human Ig gene combinatorial libraries (i.e., phage display)(Griffiths and Hoogenboom, Building an in vitro immune system: human antibodies from phage display libraries. In: Protein Engineering of Antibody Molecules for Prophylactic and Therapeutic Applications in Man. Clark, M. (Ed.), Nottingham Academic, pp 45-64 (1993); Burton and Barbas, Human Antibodies from combinatorial libraries. Id., pp 65-82). Fully human anti-stabilized Aβ protofibrillar aggregate monoclonal antibodies may also be produced using transgenic mice engineered to contain human immunoglobulin gene loci as described in PCT Patent Application WO98/24893, Jakobovits et al., published Dec. 3, 1997 (see also, Jakobovits, 1998, Exp. Opin. Invest. Drugs 7(4): 607-614). This method avoids the in vitro manipulation required with phage display technology and efficiently produces high affinity authentic human antibodies.

The method of generating an immune response to stabilized Aβ protofibrillar aggregates can be used to generate modified version of antibodies, e.g., fragments, chimera, bi-specific antibodies, and the like. Immunologically reactive fragments, such as the Fab, Fab′, or F(ab′)2 fragments are often preferable, especially in a therapeutic context, as these fragments are generally less immunogenic than the whole immunoglobulin. Further, bi-specific antibodies specific for two or more epitopes may be generated using methods generally known in the art. Further, antibody effector functions may be modified so as to enhance the therapeutic effect of stabilized Aβ protofibrillar aggregate antibodies. For example, cysteine residues may be engineered into the Fc region, permitting the formation of interchain disulfide bonds and the generation of homodimers which may have enhanced capacities for internalization, ADCC and/or complement-mediated cell killing (see, for example, Caron et al., 1992, J. Exp. Med. 176: 1191-1195; Shopes, 1992, J. Immunol. 148: 2918-2922). Homodimeric antibodies may also be generated by cross-linking techniques known in the art (e.g., Wolff et al., Cancer Res. 53: 2560-2565).

An anti stabilized Aβ protofibrillar aggregate antibody or fragment thereof of generated using the method of the invention can be labeled with a detectable marker or conjugated to a second molecule, such as a therapeutic agent (e.g., a cytotoxic agent) thereby resulting in an immunoconjugate. For example, the therapeutic agent includes, but is not limited to, an anti-plaque drug, a toxin, a radioactive agent, a cytokine, a second antibody or an enzyme. The immunoconjugate can be used for targeting the second molecule to a cell with a specific physiology. Techniques for conjugating or joining therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58(1982)).

An immune response to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

The ability of a particular antigen to stimulate a cell-mediated immunological response may be determined by a. number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Methods of measuring cell-mediated immune response include measurement of intracellular cytokines or cytokine secretion by T-cell populations, or by measurement of epitope specific T-cells (e.g., by the tetramer technique)(reviewed by McMichael, A. J., and O'Callaghan, C. A., J. Exp. Med. 187(9)1367-1371, 1998; Mcheyzer-Williams, M. G., et al, Immunol. Rev. 150:5-21, 1996; Lalvani, A., et al, J. Exp. Med. 186:859-865, 1997).

Thus, an immune response as used herein may be one which stimulates the production of CTLs, and/or the production or activation of helper T-cells. The antigen of interest may also elicit an antibody-mediated immune response. Hence, an immunological response may include one or more of the following effects: the production of antibodies by B-cells; and/or the activation of suppressor T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The dosage of the immunogen, e.g., stabilized Aβ protofibrillar aggregate, to be administered will depend on the specific immunogen used, and, in upon the age, sex, weight and condition of the recipient. The dosage should not be so large as to cause adverse side effects such as unwanted cross-reactions, generalized immunosuppression, anaphylactic reactions and the like. The dosage of the immunogen can vary depending, for example, on whether generation of the immune response is in vivo or ex vivo, whether the administration is a first administration or a booster administration, whether an adjuvant is administered with the immunogen, and, when administered in vivo, on the route of administration and the weight of the patient. Methods for determining a therapeutically effective amount of an immunogen are routine and well known in art (see Powell and Newman, Vaccine Design: The subunit and adjuvant approach (Plenum Publ. Corp.; 1994)).

For example, where stabilized Aβ protofibrillar aggregate are administered to a human, an effective amount of the stabilized Aβ protofibrillar aggregate immunogen can be in the range can be in the range of from about 0.05 μg/kg to about 50 mg/kg, usually from about 0.005 to about 5 mg/kg.

### EXAMPLE 1

### Materials and Methods

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B(1992).

Note that, as used below, the terms “Aβ-CLC aggregate” and “CLC aggregate” are used interchangeably to describe stabilized Aβ protofibrillar aggregates made using a stabilizing compound, CLC.

Materials and General Methods.

Purified wild type Aβ(1-40), (SEQ ID NO:1), and Aβ(1-42), with amino acid sequence DAEFRHDSGY EVHHQKLVFF AEDVGSNKGA IIGLMVGGVVIA (SEQ ID NO:2), and was obtained from the Keck Biotechnology Center at Yale University. Proline replacement mutants (SEQ ID NO:3-X, see Table were obtained unpurified from either the Keck Center or from the Stanford University PAN Facility. N-terminally biotinylated wild type Aβ(1-40) was obtained from the Keck Center. Analogs were purified on a C3 reverse phase column and the identity of the material confirmed by mass spectrometry on an Agilent 1100 Series LC/MSD. The LOPAC (Library of Pharmacologically Active Compounds) collection and pure samples of CLC were obtained from Sigma. The monoclonal antibody WO1 (11) was obtained from high density cell culture and used without purification. The amount of CLC in CLC-Aβ aggregates was determined by both HPLC and mass spectrometry, in both cases using a standard curve generated from analysis of a stock solution of CLC.

TABLE 1
Amino acid sequence of Aβ peptides used

| | | |
|---|---|---|
| | | SEQ ID |
| Aβ | Sequence | NO: |

| | | |
|---|---|---|
| Aβ3(1-40) | DAEFRHDSGY EVHHQKLVFF AEDVGSNKGA IIGLMVGGVV | 1 |
| Aβ3(1-42) | DAEFRHDSGY EVHHQKLVFF AEDVGSNKGA IIGLMVGGVV IA | 2 |
| F4P | DAEPRHDSGY EVHHQKLVFF AEDVGSNKGA IIGLMVGGVV | 3 |
| H6P | DAEFRPDSGY EVHHQKLVEF AEDVGSNKGA IIGLMVGGVV | 4 |
| G9P | DAEFRHDSPY EVHHQKLVFF AEDVGSNKGA IIGLMVGGVV | 5 |
| V12P | DAEFRHDSGY EPHHQKLVFE AEDVGSNKGA IIGLMVGGVV | 6 |
| H14P | DAEERHDSGY EVHPQKLVFF AEDVGSNKGA IIGLMVGGVV | 7 |
| Q15P | DAEFRHDSGY EVHHPKLVFF AEDVGSNKGA IIGLMVGGVV | 8 |
| K16P | DAEFRHDSGY EVHHQPLVFF AEDVGSNKGA IIGLMVGGVV | 9 |
| L17P | DAEFRHDSGY EVHHQKPVFF AEDVGSNKGA IIGLMVGGVV | 10 |
| V18P | DAEFRHDSGY EVHHQKLPFF AEDVGSNKGA IIGLMVGGVV | 11 |
| F19P | DAEFRHDSGY EVHHQKLVPF AEDVGSNKGA IIGLMVGGVV | 12 |
| F20P | DAEFRHDSGY EVHHQKLVFP AEDVGSNKGA IIGLMVGGVV | 13 |
| A21P | DAEFRHDSGY EVHHQKLVFF PEDVGSNKGA IIGLMVGGVV | 14 |
| E22P | DAEFRHDSGY EVHHQKLVFF APDVGSNKGA IIGLMVGGVV | 15 |
| D23P | DAEFRHDSGY EVHHQKLVFF AEPVGSNKGA IIGLMVGGVV | 16 |
| V24P | DAEFRHDSGY EVHHQKLVFF AEDPGSNKGA IIGLMVGGVV | 17 |
| G25P | DAEFRHDSGY EVHHQKLVFF AEDVPSNKGA IIGLMVGGVV | 18 |
| S26P | DAEFRHDSGY EVHHQKLVFF AEDVGPNKGA IIGLMVGGVV | 19 |
| N27P | DAEFRHDSGY EVHHQKLVFF AEDVGSPKGA IIGLMVGGVV | 20 |
| K28P | DAEFRHDSGY EVHHQKLVFF AEDVGSNPGA IIGLMVGGVV | 21 |
| G29P | DAEFRHDSGY EVHHQKLVFF AEDVGSNKPA IIGLMVGGVV | 22 |
| A30P | DAEFRHDSGY EVHHQKLVFF AEDVGSNKGP IIGLMVGGVV | 23 |
| I31P | DAEFRHDSGY EVHHQKLVFF AEDVGSNKGA PIGLMVGGVV | 24 |
| I32P | DAEFRHDSGY EVHHQKLVFF AEDVGSNKGA IPGLMVGGVV | 25 |
| G33P | DAEFRHDSGY EVHHQKLVFF AEDVGSNKGA IIPLMVGGVV | 26 |
| L34P | DAEFRHDSGY EVHHQKLVFF AEDVGSNKGA IIGPMVGGVV | 27 |
| M35P | DAEFRHDSGY EVHHQKLVFF AEDVGSNKGA IIGLPVGGVV | 28 |
| V36P | DAEFRHDSGY EVHHQKLVFF AEDVGSNKGA IIGLMPGGVV | 29 |
| G37P | DAEFRHDSGY EVHHQKLVFF AEDVGSNKGA IIGLMVPGVV | 30 |
| G38P | DAEFRHDSGY EVHHQKLVFF AEDVGSNKGA IIGLMVGPVV | 31 |
| V39P | DAEFRHDSGY EVHHQKLVFF AEDVGSNKGA IIGLMVGGPV | 32 |
| P2 | DAEFRHDSGY EVHHQKLVFF AEPVGSNKGP IIGLMVGGVV | 33 |
| P4 | DAEFRHDSPY EVHHQKLVFF AEPVGSNKGP IIGLMVPGVV | 34 |
| β2 | DAEFRHDSGY EVHHQKLVPF AEDVGSNKGA IPGLMVGGVV | 35 |

Microtiterplate Elongation and Screening Assays. The Aβ(1-40) microplate elongation assay has been described (12). For screening the compound library, plates were coated with 100 ng Aβ(1-40) fibrils and washed. The assay was initiated by adding to each well, in order, 80 μl assay buffer [PBSA (phosphate-buffered saline containing 0.05% (w/v) sodium azide) containing 0.05% Tween 20], 10 μl of a 1 mM solution of test compound in DMSO, and 10 μl 100 nM biotinyl-Aβ in assay buffer. Plates were incubated 30 mins at 37° C., then washed and the signal developed as described using a streptavidin-europium linked fluorescence assay (12, 13). Each microplate collection of compounds was assayed in triplicate, and results reported as % inhibition compared to control wells lacking test compounds.

Solution Phase Aggregation Assays.

Aβ(1-40) peptides were disaggregated just prior to use, as described previously (12, 14). Peptides (50 μM in fibril formation reactions, approximately 30 μM in CLC aggregation reactions) were incubated in 50 μM PBSA at 37° C. Reaction progress was followed by ThT fluorescence and/or quantitative HPLC on centrifugation (30 mins, 315,000×g) supernatants of reaction aliquots (12, 15). The Cr value is the concentration of Aβ in solution when this value stops changing with time. For amyloid fibril growth, this value represents a position of dynamic equilibrium and is the reciprocal of the growth equilibrium constant for the amyloid fibrils (15); for the CLC-induced aggregation, the value is a more qualitative measure of the stability of the Aβ-CLC aggregates. Samples of aggregates were stored at −80° C. after snap-freezing of washed pellets. Reactions with CLC contained 10% DMSO from the CLC stock solution.

Hydrogen-Deuterium Exchange by Mass Spectrometry (HX-MS).

The set-up and protocols used for HX-MS experiments were as previously described (8, 16, 17). All aggregates and fibrils were collected by centrifugation, washed, and resuspended in either protonated or deuterated 2 mM Tris buffer. The sample solution and sample processing solvent containing water, acetonitrile and formic acid were mixed on-line in a T tube prior to electrospray ionization. All of the HX data presented here were collected using a Micromass Quattro II spectrometer, and corrected for artifactual exchange of rapidly exchanging side-chain and terminal protons. Data for CLC-Aβ. aggregates were also corrected for artifactual forward and backward exchange of backbone amide protons, as described (17), using exchange data from fully deuterated CLC-Aβ(1-40).

Antibody Binding Assays.

Aβ fibrils and CLC-Aβ aggregates were immobilized onto 96-well high binding microtiter plate (Costar) by adding 50 μL of a 2 ng/μL solution of the fibrils/protofibrils in PBSA to each well and allowing the plate to dry by overnight incubation unsealed at 37° C. The plates were then washed twice with PBSA containing 0.05% Tween 20, blocked with 1% gelatin in PBSA, and used to determine binding to aggregates of the antibody WO1 or control mouse myeloma IgM (CalBiochem) as described (11).

### EXAMPLE 2

### Library Screening

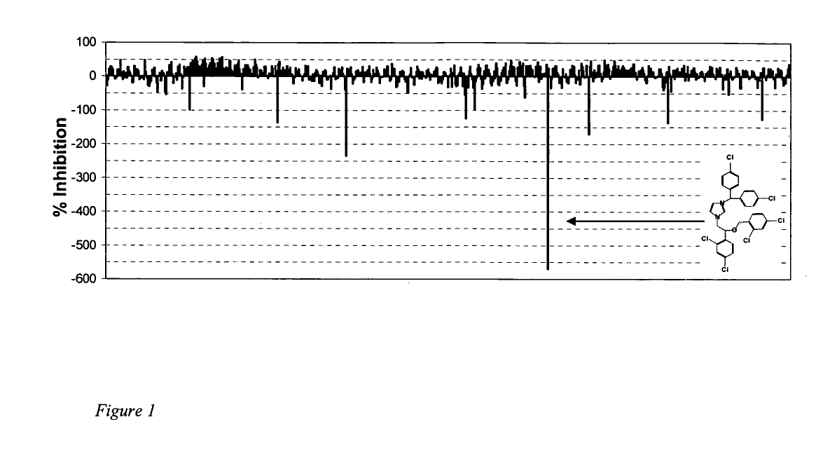

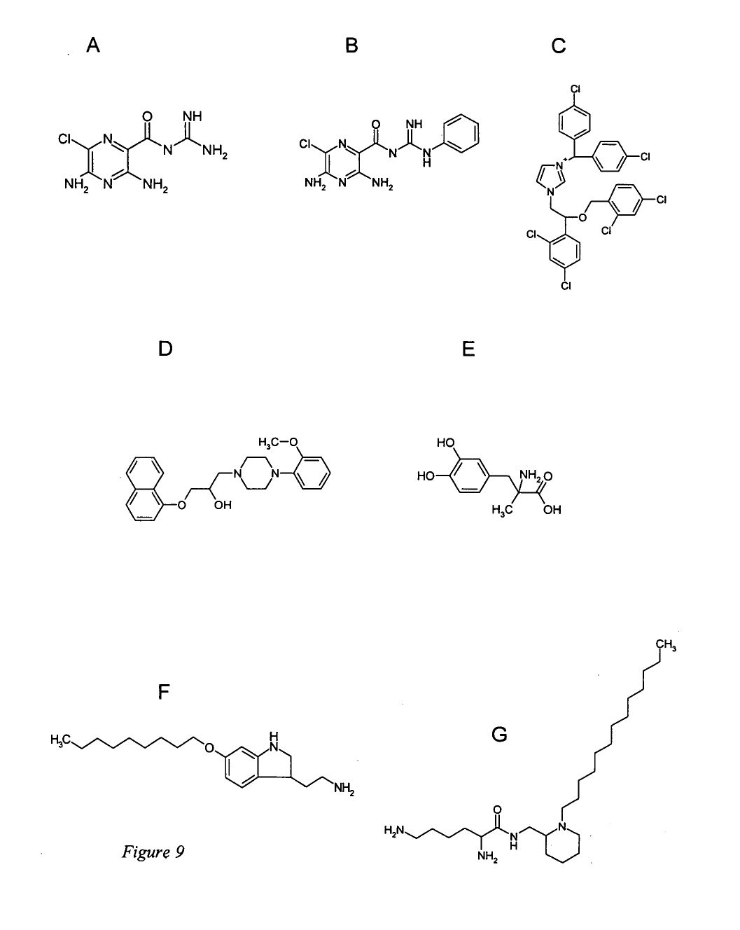

We screened a small library of 640 compounds, the LOPAC library (Sigma), using a microplate-based Aβ elongation assay. In this assay, plastic wells containing immobilized fibrils are incubated with a biotin-tagged monomer in the presence or absence of potential modifiers (13, 18). We found a number of compounds with potent abilities to enhance deposition of the biotin-tagged Aβ(1-40) (FIG. 1). Seven of the compounds were selected for further analysis:—amiloride HCl, phenamil methane sulfonate, naftopidil dihydrochloride, NPC 15437 dihydrochloride, calmidazolium chloride (R24571), 5-(nonoxyl)-tryptamine hydrogen oxalate, and L-α-methyl DOPA. The compounds have a wide variety of structures and biochemical properties. The structures of these seven compounds are shown in FIG. 9, along with the percent stimulation observed in the experiments.

The most potent of these compounds is calmidazolium chloride (CLC, FIG. 1 insert), which at 100 μM stimulates the deposition of about six times as much Aβ compared to the elongation reaction in the absence of added compound.

Four compounds—calmidazolium chloride, NPC 15437, phenamil methane sulfonate, and amiloride HCl were chosen for secondary screening, e.g., assaying for formation of stabilized Aβ protofibrillar aggregates in the absence of seeding Aβ amyloid fibrils. All four compounds to be retested were purchased from Sigma. Three of the four compounds (NPC 15437, phenamil methane sulfonate, and amiloride HCl) demonstrated sub-optimal activity in enhancing aggregate formation in the absence of seeding Aβ amyloid fibrils (data not shown). As described in more detail below, CLC was shown to be a potent stabilizing compound.

### EXAMPLE 3

### Characterization of CLC Stabilized Protofibrillar Aggregates

Although clearly distinct structures, Aβ mature fibrils exhibit a number of similarities to protofibrils and CLC-Aβ aggregates, including a fundamental fibrillar character in the EM, a shared epitope for the antibody WO1, and common possession of a subset of unusually stable H-bonds. Both mature amyloid fibrils and CLC aggregates appear to be assembled from very similar conformations of the peptide, as revealed by scanning proline mutagenesis, which can reveal sites of restrictive peptide backbone geometry within fibrils (15, 22).

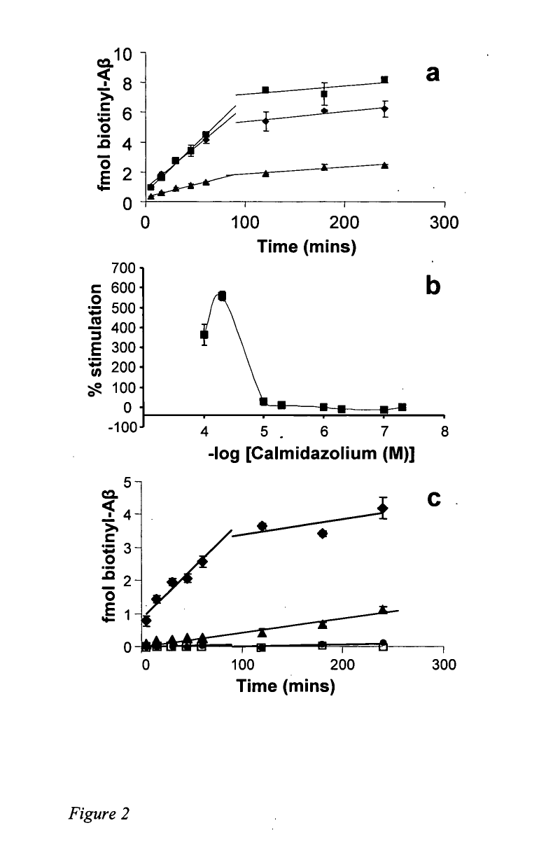

We characterized the activity of CLC in the microplate assay in more detail. The time course of Aβ deposition in the presence of CLC yields a pattern similar to the reaction without added compound (FIG. 2a), suggesting a simple acceleration of the normal mechanism. However, when the assay is repeated on a microplate lacking Aβ fibrils, the dramatic deposition of biotinylated Aβ in the presence of CLC was maintained (FIG. 2a), suggesting a direct ability of CLC to interact with soluble Aβ and thereby produce rapid deposition. Dose response analysis shows that this stimulation of the deposition of 10 nM Aβ occurs at concentrations of CLC in the 10-100 μM range (FIG. 2b).

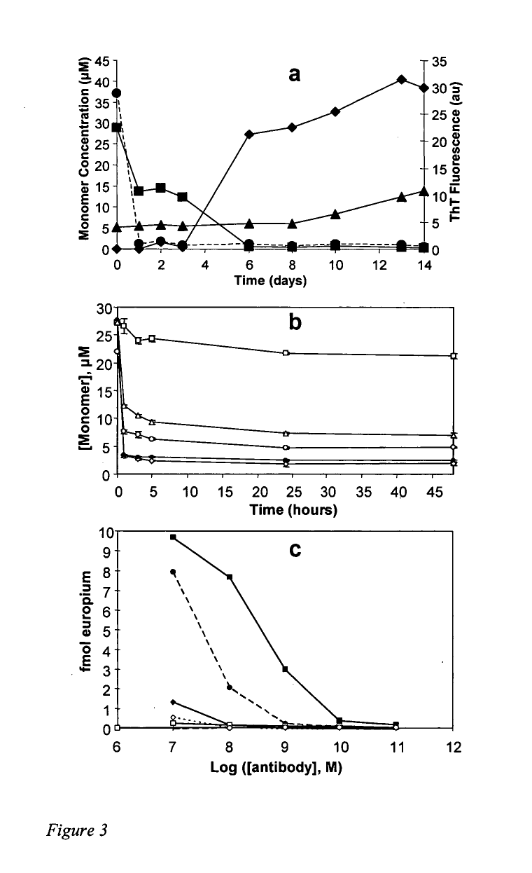

To confirm the microtiter plate assay, we carried out solution phase experiments using unlabelled Aβ(1-40). Aβ incubated alone exhibits a thioflavin T (ThT) lag phase of several days before progressing to a rapid amyloid fibril growth phase (FIG. 3a). During the ThT lag phase, the amount of soluble, monomeric Aβ is reduced by about a factor of two, consistent with the formation of protofibrillar structures with low ThT response (2). In contrast, in the presence of 100 μM CLC, Aβ(1-40) is rapidly converted to a pelletable aggregate, so that within 1-2 hrs only 2-3 μM Aβ remains in solution after centrifugation (FIG. 3a,b); when these aggregates are isolated by sedimentation, they are found to contain a ratio of 4 moles of CLC per mole of Aβ(1-40) (results of independent analyses against both HPLC and MS standard curves). The significance of this ratio is not clear. Interestingly, this deposition is not associated with any apparent ThT signal (FIG. 3a); the low ThT signal obtained at reaction initiation persists for days, then gradually increases over a period of 14 days to reach a level about 20% of that observed for the same weight of mature amyloid fibrils (FIG. 3a).

The low ThT response indicates a resemblance between the CLC-Aβ aggregates and normal protofibrils. FIG. 3a shows that the pelletable Aβ that accumulates both in the early stages of spontaneous amyloid growth in the absence of CLC, and the CLC-Aβ aggregates, give similar, low ThT responses. These observations are in qualitative agreement with previous ThT measurements of Aβ protofibrils isolated in the lag phase of normal Aβ amyloid growth (2).

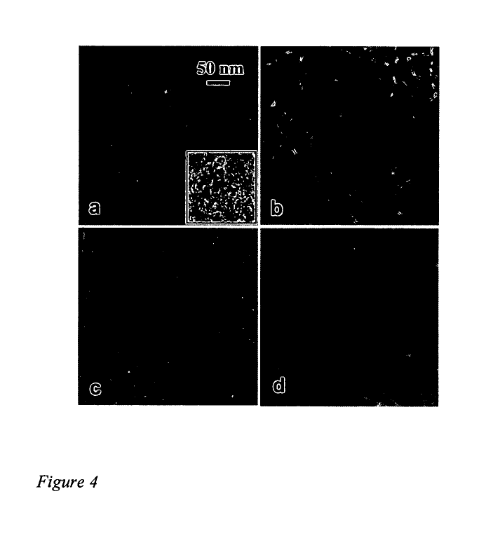

EM suggests that the aggregates induced by CLC more resemble protofibrils than mature fibrils. Thus, in an incubation of Aβ(1-40) without CLC, aggregates collected at day 2 (within the ThT lag phase) are curvilinear, rough-edged protofibrils (FIG. 4a), as reported previously (1, 2, 7, 19-21). Aggregates collected from the same reaction at day 14 are typical amyloid fibrils (FIG. 4b). Aggregates isolated from the two-day reaction in the presence of CLC exhibit large spheroids (not shown) that, when rigorously vortexed, yield EM images showing a dense lawn of structures (FIG. 4c) resembling normal protofibrils (FIG. 4a). Aggregates isolated from a 14-day reaction contain similar spheroidal clusters of protofibrils, accompanied by long fibril-like ribbons (FIG. 4d). The spheroidal structures are around 5 nM in diameter and 50-100 nM in length; each aggregate has about 100 monomers. This apparent partial conversion of protofibrils to fibrils after 14 days incubation is consistent with the increased ThT signal observed for this reaction (FIG. 3a). Although these fibrillar structures are associated with the globular protofibril clusters on the EM grids, it is not possible to say whether the fibrils grow out of the protofibril clusters or associate with them post-synthetically.

That CLC aggregates exhibit fine structure is highly reminiscent of a particular type of protofibril often observed by EM (2, 19, 20) and AFM (1, 7, 21). Normal Aβ protofibrils incubated in the absence of excess Aβ monomer tend to dissociate (7); while CLC stabilizes the Aβ aggregates, minimizing dissociation, these aggregates are incapable of elongating via monomer addition (FIG. 2c), supporting a resemblance to normal protofibrils.

Biochemical analysis of the CLC aggregates is consistent with the structures observed in the EM. In one analysis, aggregates were collected by centrifugation at 2 and 14 days from Aβ(1-40) incubations done in the absence or presence of CLC. The concentrations of the aggregates were calibrated by HPLC analysis of dissolved aliquots, and equal weights of each immobilized onto microtiter plate wells. Neither the oligomers grown from Aβ alone (FIG. 4a), nor the CLC-Aβ aggregates collected after 2 days (FIG. 4c), were effective seeds for elongation by monomeric Aβ(1-40) (FIG. 2c). This inability to seed elongation is consistent with the known instability of Aβ protofibrils, which tend to dissociate when removed from high concentrations of Aβ (7). In contrast, mature Aβ amyloid fibrils (FIG. 4b) exhibit high elongation seeding ability, while the 14 day CLC-Aβ aggregates (FIG. 4d) give relatively low seeding activity consistent with EM and ThT evidence of their being a mixture of fibrils and protofibrils.

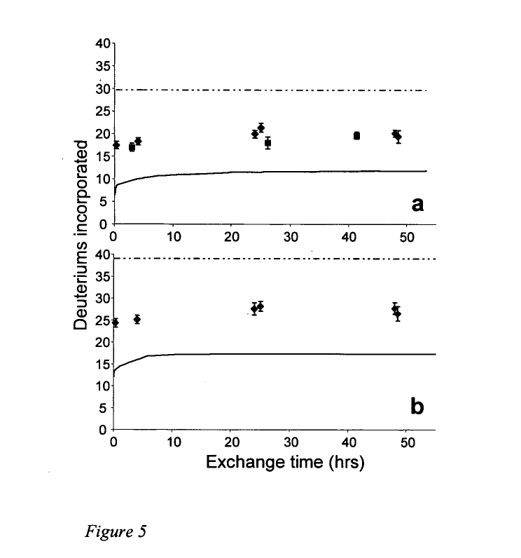

The CLC-Aβ aggregates also are very similar to Aβ protofibrils in their HX behavior. Previously we showed that about half of the 39 backbone amide hydrogens of Aβ(1-40) are very stably protected in HX of mature amyloid fibrils (16, 17), while substantially fewer of these amide hydrogens are protected in Aβ(1-40) protofibrils (8). FIG. 5a shows that the degree of protection in the CLC-Aβ aggregates is exactly the same as that seen previously for authentic Aβ protofibrils isolated from normal fibril formation reactions (8). The coincidence between the kinetics and extents of protection provides further evidence that CLC-Aβ aggregates are very similar to the protofibrils normally observed during Aβ fibril assembly.

In our previous work on HX of protofibrils, it was only possible to correct the HX data for rapidly exchanging side chain protons (8). To obtain an absolute number for protection in protofibrils, it is also necessary to correct the data for artifactual forward and backward exchange into the backbone amide protons (17). We prepared CLC aggregates from fully deuterated Aβ(1-40) and determined their back exchange rate which, along with forward exchange data from protonated aggregates, was used to correct the experimental data (17). The fully corrected kinetic points are plotted in FIG. 5b, which also shows the fitted data for fully corrected amyloid fibril HX as well as the total number of exchangeable backbone amide protons. The data indicate that the CLC stabilized Aβ(1-40) aggregates contain only about 12 highly protected backbone amide protons after two days of exchange time, in contrast to about 22 for Aβ(1-40) fibrils. Previous studies demonstrated the ability of HX to reproducibly distinguish among a variety of Aβ assemblies (8). The close match in both exchange kinetics and final amplitude between protofibrils and CLC-Aβ aggregates strongly suggests a structural relationship.

That these novel aggregates also share some structural features with mature fibrils is suggested by the ability of the CLC-Aβ aggregates to bind, with about 10-fold reduced affinity, to the amyloid-specific antibody WO1 (11), as shown in FIG. 3c. This antibody has been shown to recognize amyloid fibrils from many different proteins, while not binding to the monomeric precursor proteins. It has not previously been studied with protofibrils, partially because of difficulties maintaining protofibril integrity during binding assays. The results suggest that the amyloid epitope detected by the antibody is already present in the CLC-Aβ aggregate and hence in protofibrils.

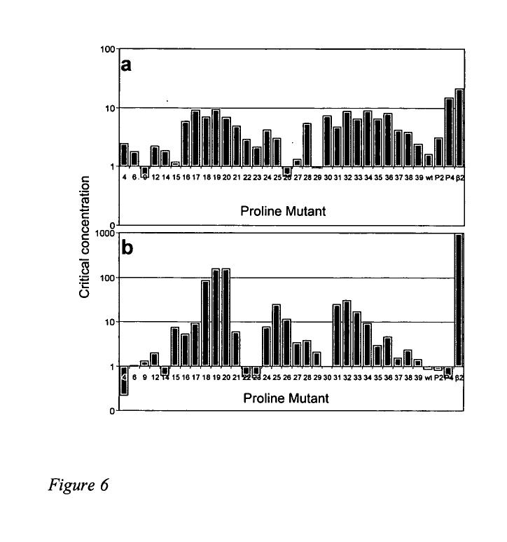

To further probe the relationship between the conformation of Aβ in fibrils and protofibrils, we turned to scanning proline mutagenesis analysis, which we employed previously to provide insights into the Aβ conformation in mature amyloid fibrils (15). Proline point mutants of Aβ(1-40) were treated with CLC and the aggregation reactions followed until the amount of Aβ in solution after centrifugation was unchanged (FIG. 3b). The amino acid sequences of the proline point mutants are shown in Table 1. These values, which represent the degree to which proline at each sequence position destabilizes the aggregate (15), are plotted in the bar graph in FIG. 6a. The corresponding values for amyloid fibril formation by the same Aβ(1-40) mutants, most of which were previously reported, (15), are shown in FIG. 6b.

The comparison shows both similarities and differences in the sensitivities of the two aggregates (mature amyloid fibrils and CLC aggregates) to proline substitution. In both aggregates, replacement of residues in the N-terminal region is negligibly destabilizing, while proline substitution at many other positions in the 15-40 segment, especially the hydrophobic clusters at positions 16-21 and 31-36, are destabilizing. Illustrative of this, a double Pro mutant at positions 19 and 32 produces the most destabilization of any mutant for both mature fibrils and CLC aggregates. FIG. 6 also shows some significant differences between fibrils and CLC aggregates. In a striking example, a tetra-substituted mutant peptide (designated P4 in the figure), with prolines at positions 9, 23, 30 and 37, makes amyloid fibrils of the same stability as wild type Aβ(1-40), but produces CLC aggregates that are significantly destabilized compared to WT. The source of this destabilization is apparent in closer examination of the single point mutant data, which shows that prolines at positions 30 and 37 significantly destabilize CLC aggregates but not fibrils. Conversely, Pro replacement at residues 24-28 more strongly affects amyloid fibril formation than CLC aggregate formation.

We found a substantial overlap in the destabilization profiles for the amyloid fibrils and CLC aggregate forms in the scanning proline mutagenesis analysis (FIG. 6). This suggests that the structure providing HX protection to protofibrils lies in H-bonded β-sheet involving the same hydrophobic patches 16-21 and 31-36 involved in fibril structure. Significantly, in both fibrils and CLC aggregates, the N-terminal 14 residues and the C-terminal 3-4 residues seem uninvolved in the kind of rigid structure disrupted by proline substitution (FIG. 6). The involvement of residues in the 21-30 segment in rigid structure, however, differs between these two aggregated states. The data suggests that the CLC aggregate may involve Aβ in more of a simple extended hairpin structure with a long, relatively flexible loop, involving residues 22-29, spanning the two β-structure segments. The major structural difference in the Aβ sequence when it adopts amyloid fibril structure may thus be a better definition of structure in the 22-29 segment. This is supported by pepsin-HX analysis of the CLC-Aβ protofibrils (data not shown).

It should be pointed out that, while WT Aβ(1-40) exhibits very similar Cᵣ values for both fibril (0.8 μM) and protofibril (1.6 μM) formation, the stability of CLC aggregates is augmented by the binding energy of the CLC component. We assume that it is for this reason that the destabilizing effects of proline replacement are significantly dampened in the CLC-Aβ aggregates compared to amyloid fibrils (FIG. 6b). The overall resemblance of the profiles for fibrils and CLC aggregates in FIG. 6 strongly suggests that the over-riding structural bases for proline destabilization are similar for the two aggregated states of Aβ.

### EXAMPLE 4

### AB(1-42)/Calmidazolium Studies

The interaction of calmidazolium chloride (CLC) with AP(1-42) was examined. The results show a very close parallel to the results with the 1-40 peptide, suggesting that the CLC-Aβ(1-42) aggregates are stabilized protofibrils and that these differ immunochemically from amyloid fibrils.

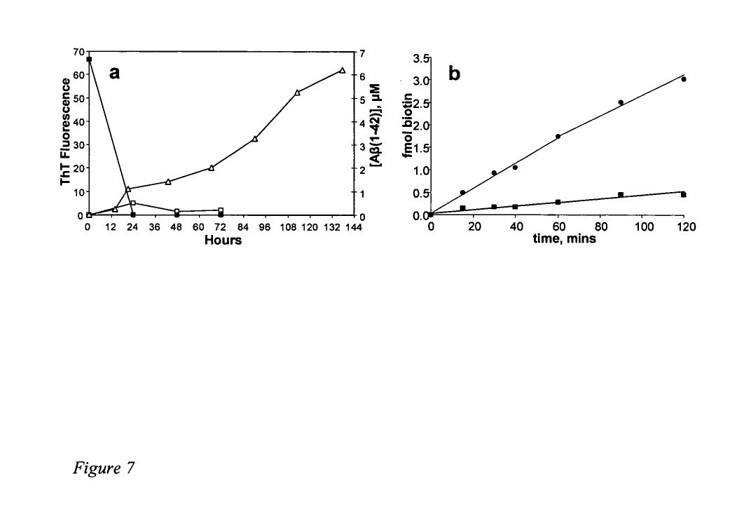

FIG. 7a shows the production of Aβ(1-42)-CLC aggregates. This is similar to FIG. 3a and the 1-40 peptide. It shows very rapid deposition of Aβ(1-42) to an aggregate that has very little ThT signal relative to the Aβ(1-42) fibrils.

FIG. 7b shows that most of the CLC-Aβ(1-42) aggregates are not amyloid, given the very poor ability of this preparation to serve as a substrate for amyloid fibril elongation. This is further support that these are stabilized protofibrils. This study is similar to that shown in FIG. 2c and the 1-40 peptide.

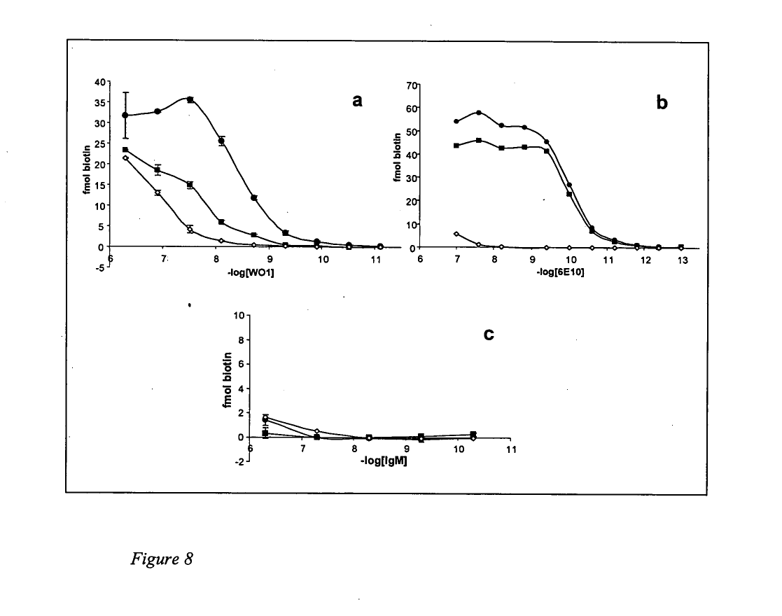

FIG. 8 summarizes experiments with the antibody WO1 and control antibodies to further characterize the CLC-Aβ(1-42) aggregates. FIG. 8a shows the binding curves for WO1. It can be seen that Aβ(1-42) amyloid fibrils exhibit relatively strong and cooperative binding, as reported previously for 1-40 fibrils. The CLC-Aβ(1-42) aggregates give a much less cooperative binding curve that is also displaced to a weaker binding midpoint (higher MAb concentration). This is not due to any difference in the amount of Aβ(1-42) aggregate on the plate for the two aggregate types, since the Aβ N-terminus linear epitope antibody 6E10 (Chemicon) picks up both aggregates at about the same amount (FIG. 8b). The binding of WO1 to CLC-Aβ(1-42) aggregates is significant, as can be seen by comparing them (FIG. 8a) to the corresponding binding curves generated by a control IgM (mouse myeloma IgM, Calbiochem) (FIG. 8c). The FIG. 8a data is similar to that for Aβ(1-40)-CLC aggregates (FIG. 3c).

The results described here show that the aggregated structures formed from Aβ(1-40) and Aβ(1-42) in the presence of CLC resemble the protofibrillar structures normally observed during the formation of mature amyloid fibrils from this peptide.

### EXAMPLE 5

### Discussion

Although these experiments give us a much clearer picture of protofibril structure, they cannot address the question of the relevance of protofibrils in the amyloid fibril assembly mechanism. The fact that CLC-Aβ aggregates partially transform into an amyloid-like structure on prolonged incubation, coupled with the physical proximity of spheroidal CLC-Aβ aggregates in EM grids (FIG. 4d), is consistent with a precursor-product relationship between protofibrils and fibrils. Details of HX studies on isolated protofibrils previously led to a similar suggestion (8). However, it remains possible that incubated CLC-Aβ aggregates could produce fibrils by dissociation and reassembly of monomeric Aβ, while the physical proximity on grids, as shown in FIG. 4d, could be an artifact of aggregate stickiness. It is also possible that the fundamental characteristics of the Aβ sequence, which displays β-extended chain structure propensity in regions 17-21 and 31-36 even as a soluble monomer (23), might influence the structures of both aggregates independently. Recent data showing that under some conditions Sup35 fibril formation involves a small critical nucleus and growth by monomer addition (24) suggests that more work needs to be done to establish the role of protofibrils in amyloid assembly.

The ability of CLC to accelerate protofibril formation and stabilize protofibril structure was discovered during a random screen for modulators of Aβ fibril elongation. Other random screening efforts have unveiled compounds that stimulate formation of alternatively aggregated forms of Aβ and hence retard fibril formation (25-27). Although there are no strong structural similarities, these compounds share with CLC the combination of a basic nitrogen moiety with substituted aromatic or heteroaromatic groups. It is possible that aromatic interactions between these compounds and Phe residues 19 and 20 may contribute to the ability of compounds like CLC to stabilize Aβ protofibril structure. It has been speculated that phenylalanine interactions may promote amyloid formation in at least some amyloidogenic sequences (28, 29). In this regard, it may be relevant that the stabilization of protofibril structure is also a property of the “Arctic” mutant of Aβ (30), which involves a Glu->Gly mutation at position 22 of Aβ, directly adjacent to the Phe-Phe pair at positions 19 and 20. Interestingly, treatment of Aβ peptides with organic solvents can also induce formation of non-fibrillar aggregates (31). The relationship between any of these alternatively aggregated structures and the CLC-Aβ aggregates described here is not clear.

As shown here, compounds like CLC can prove useful in facilitating detailed studies of protofibril structure. Compounds that stabilize protofibrillar aggregate structures can also have therapeutic value. Although recent evidence suggests that oligomers and protofibrils may be the toxic entities in at least some amyloid diseases (4, 5), it is possible that a drug that facilitates sequestration of monomeric Aβ into stabilized protofibrils may provide therapeutic advantage, protecting cells while giving the body time to remove or inactivate these peptides.

In addition, chemically stabilized protofibrillar structures can be used as tools to generate and/or screen for antibodies specific for protofibrils using antibody methods well known to one of skill in the art. Antibodies specific for protofibrils can be used as diagnostic tools or as therapeutics in the diagnosis or treatment of, e.g., Alzheimer's disease. Finally, chemically stabilized protofibrillar structures can be used in the preparation of therapeutic or prophylactic vaccines.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES

1. Lambert, M. P., Barlow, A. K., Chromy, B. A., Edwards, C., Freed, R., Liosatos, M., Morgan, T. E., Rozovsky, I., Trommer, B., Viola, K. L., Wals, P., Zhang, C., Finch, C. E., Krafft, G. A. & Klein, W. L. (1998) Proc Natl Acad Sci USA 95, 6448-53.
2. Walsh, D. M., Hartley, D. M., Kusumoto, Y., Fezoui, Y., Condron, M. M., Lomakin, A., Benedek, G. B., Selkoe, D. J. & Teplow, D. B. (1999) J Biol Chem 274, 25945-25952.
3. Harper, J. D., Wong, S. S., Lieber, C. M. & Lansbury, P. T., Jr. (1999) Biochemistry 38, 8972-80.

4. Kirkitadze, M. D., Bitan, G. & Teplow, D. B. (2002) J Neurosci Res 69, 567-77.

5. Caughey, B. & Lansbury, P. T. (2003) Annu Rev Neurosci 26, 267-98.
6. Serio, T. R., Cashikar, A. G., Kowal, A. S., Sawicki, G. J., Moslehi, J. J., Serpell, L., Arnsdorf, M. F. & Lindquist, S. L. (2000) Science 289, 1317-21.
7. Harper, J. D., Wong, S. S., Lieber, C. M. & Lansbury, P. T. (1997) Chem Biol 4, 119-25.
8. Kheterpal, I., Lashuel, H. A., Hartley, D. M., Walz, T., Lansbury, P. T., Jr. & Wetzel, R. (2003) Biochemistry 42, 14092-8.
9. Bitan, G. & Teplow, D. B. (2004) Acc Chem Res 37, 357-64.
10. Findeis, M. A. (2000) Biochim Biophys Acta 1502, 76-84.
11. O'Nuallain, B. & Wetzel, R. (2002) Proc Natl Acad Sci USA 99, 1485-1490.
12. O'Nuallain, B., Williams, A. D., Westermark, P. & Wetzel, R. (2004) J Biol Chem 279, 17490-17499.
13. Berthelier, V., Hamilton, J. B., Chen, S. & Wetzel, R. (2001) Anal Biochem 295, 227-36.
14. Kheterpal, I., Williams, A., Murphy, C., Bledsoe, B. & Wetzel, R. (2001) Biochem. 40, 11757-11767.
15. Williams, A. D., Portelius, E., Kheterpal, I., Guo, J. T., Cook, K. D., Xu, Y. & Wetzel, R. (2004) J Mol Biol 335, 833-42.
16. Kheterpal, I., Zhou, S., Cook, K. D. & Wetzel, R. (2000) Proc Natl Acad Sci USA 97, 13597-601.
17. Kheterpal, I., Wetzel, R. & Cook, K. (2003) Protein Sci 12, 635-643.
18. Esler, W. P., Stimson, E. R., Ghilardi, J. R., Felix, A. M., Lu, Y. A., Vinters, H. V., Mantyh, P. W. & Maggio, J. E. (1997) Nat Biotechnol 15, 258-63.
19. Nybo, M., Svehag, S. -E. & Nielsen, E. H. (1999) Scand. J. Immunol. 49,219-223.
20. Lashuel, H. A., Hartley, D. M., Petre, B. M., Wall, J. S., Simon, M. N., Walz, T. & Lansbury, P. T., Jr. (2003) J Mol Biol 332, 795-808.
21. Chromy, B. A., Nowak, R. J., Lambert, M. P., Viola, K. L., Chang, L., Velasco, P. T., Jones, B. W., Fernandez, S. J., Lacor, P. N., Horowitz, P., Finch, C. E., Krafft, G. A. & Klein, W. L. (2003) Biochemistry 42, 12749-60.
22. Wood, S. J., Wetzel, R., Martin, J. D. & Hurle, M. R. (1995) Biochem. 34, 724-730.
23. Hou, L., Shao, H., Zhang, Y., Li, H., Menon, N. K., Neuhaus, E. B., Brewer, J. M., Byeon, I. J., Ray, D. G., Vitek, M. P., Iwashita, T., Makula, R. A., Przybyla, A. B. & Zagorski, M. G. (2004) J Am Chem Soc 126, 1992-2005.
24. Collins, S. R., Douglass, A., Vale, R. D. & Weissman, J. S. (2004) PLoS 2, 1582-1590.
25. Bohrrmann, B., Adrian, M., Dubochet, J., Kuner, P., Muller, F., Huber, W., Nordstedt, C. & Dobeli, H. (2000) J Struct Biol 130, 232-46.
26. Lashuel, H. A., Hartley, D. M., Balakhaneh, D., Aggarwal, A., Teichberg, S. & Callaway, D. J. (2002) J Biol Chem 277, 42881-90.
27. Blanchard, B. J., Chen, A., Rozeboom, L. M., Stafford, K. A., Weigele, P. & Ingram, V. M. (2004) Proc Natl Acad Sci USA 101, 14326-32.
28. Azriel, R. & Gazit, E. (2001) J Biol Chem 276, 34156-61.
29. Zanuy, D., Porat, Y., Gazit, E. & Nussinov, R. (2004) Structure (Camb) 12, 439-55.
30. Nilsberth, C., Westlind-Danielsson, A., Eckman, C. B., Condron, M. M., Axelman, K., Forsell, C., Stenh, C., Luthman, J., Teplow, D. B., Younkin, S. G., Naslund, J. & Lannfelt, L. (2001) Nat Neurosci 4, 887-93.
31. Nichols, M. R., Moss, M. A., Reed, D. K., Cratic-McDaniel, S., Hoh, J. H. & Rosenberry, T. L. (2005) J Biol Chem 280, 2471-80.

What is claimed is:

1. A method for producing a stabilized Aβ protofibrillar aggregate comprising contacting a plurality of Aβ peptide with a stabilizing compound, wherein said stabilizing compound stabilizes a protofibrillar aggregate form of the peptide in comparison to the peptide not in contact with the stabilizing compound.

2. The method of claim 1, wherein stabilization is determined using a sedimentation assay comprising centrifugation and analytical HPLC.

3. The method of claim 1, wherein the Aβ protofibrillar aggregate structure is detectably different from that of amyloid fibrils.

4. The method of claim 3, wherein the difference between the stabilized Aβ protofibrillar aggregate amyloid fibrils is detected according to the result of an assay selected from the group consisting of electron microscopy, thioflavin T response, ability to seed aggregation in the presence of Aβ peptide, and protection of backbone amide hydrogens from hydrogen-deuterium exchange.

5. The method of claim 1, wherein said peptide is Aβ (1-40).

6. The method of claim 1, wherein said peptide is Aβ (1-42).

7. The method of claim 1, wherein prior to contact with the stabilizing compound, no amyloid fibril is present.

8. The method of claim 1, wherein said stabilizing compound comprises calmidazolium chloride (CLC).

9. The method of claim 8, wherein said compound consists of calmidazolium chloride (CLC).

10. The method of claim 1, comprising contacting Aβ (1-40) peptide with calmidazolium chloride (CLC).

11. The stabilized Aβ protofibrillar aggregate produced by the method of claim 1.

12. A compound comprising an Aβ protofibrillar aggregate stabilized by a stabilizing compound.

13. The compound of claim 12, wherein Aβ(1-40) is stabilized by CLC.

14. A pharmaceutical composition comprising the stabilized Aβ protofibrillar aggregate of claim

15. A method of screening for a stabilizing compound comprising contacting an Aβ peptide with a test compound and detecting the presence or absence of a stabilized Aβ protofibrillar aggregate, wherein the presence of the stabilized Aβ protofibrillar aggregate indicates that the test compound is a stabilizing compound.

16. The method of claim 15, wherein said method is performed in the absence of amyloid fibrils.

17. The method of claim 15, wherein said method is performed in the presence of amyloid fibrils.

18. A method of screening for a binding reagent specific for a stabilized Aβ protofibrillar aggregate, said method comprising contacting a test binding reagent with the stabilized Aβ protofibrillar aggregate and detecting the presence or absence of specific binding to the stabilized Aβ protofibrillar aggregate, wherein specific binding indicates the test binding reagent is a binding reagent specific for a stabilized Aβ protofibrillar aggregate.

19. The method of claim 18, wherein said test binding reagent is selected from the group consisting of an antibody, a polypeptide, or a nucleic acid.

20. The method of claim 19, wherein the test binding reagent is an antibody.

21. The method of claim 18, wherein the Aβ protofibrillar aggregate is immobilized on a solid support.

22. The method of claim 18, wherein the Aβ protofibrillar aggregate comprises CLC.

23. The method of claim 18, wherein the Aβ protofibrillar aggregate comprises Aβ (1-40) or Aβ (1-42).

24. A method of generating an immune response in an animal comprising administering an effective amount of a stabilized Aβ protofibrillar aggregate.

25. The method of claim 24, wherein the stabilized Aβ protofibrillar aggregate comprises CLC and Aβ (1-40).

26. The method of claim 24, wherein said animal is a human.

27. The method of claim 24, wherein said animal is a rabbit or mouse.

28. The method of claim 27, further comprising the step of isolating an antibody that specifically binds the stabilized Aβ protofibrillar aggregate.

29. The method of claim 28, wherein the antibody is polyclonal or monoclonal.
